(19) United States
(12) Patent Application Publication
(10) Pub. No.: US 2016/0000949 A1
(43) Pub. Date: Jan. 7, 2016

(54) APPARATUS FOR THE GENERATION OF LOW-ENERGY X-RAYS

(71) Applicants: Yuri UDALOV, (US); Sergey MITKO, (US); ENXRAY LIMITED, Liverpool (GB)

(72) Inventors: Yuri UDALOV, Liverpool (GB); Sergey MITKO, Liverpool (GB)

(21) Appl. No.: 14/768,459

(22) PCT Filed: Feb. 25, 2014
(86) PCT No.: PCT/GB2014/050559

§ 371 Date: Aug. 18, 2015

(30) Foreign Application Priority Data
Feb. 27, 2013 (GB) 1303517.5

(51) Int. Cl. A61L 2/08; H01J 35/18; G01N 23/02; H01J 35/06; H01J 35/08

(52) U.S. Cl. CPC A61L 2/082; H01J 35/06; H01J 35/08; G01N 23/02; H01J 35/18; H01J 2235/06; H01J 2235/08; H01J 2235/18

(57) ABSTRACT

An X-ray source for producing soft X-rays, the X-ray source comprising: a cathode having an electron-emitting structure supported by a support structure, the electron-emitting structure being at least partially transparent to X-rays within a region bounded by the support structure; an anode having an X-ray emitting surface parallel to the electron-emitting structure of the cathode; and an electrically insulating spacer arranged between the anode and the cathode; wherein the electron-emitting structure of the cathode and the X-ray emitting surface of the anode are arranged such that, in use, the electron-emitting structure is operable to bombard the anode with electrons, causing X-rays to be emitted from the X-ray emitting surface and to pass through the cathode; and wherein the insulating spacer is arranged between the anode and the support structure of the cathode and projects beyond the support structure, across part of the anode, into the said region.

### FIELD OF THE INVENTION

The present invention relates to apparatus for the generation of X-rays. It is particularly applicable, but by no means limited, to low-energy X-ray generators for sterilising medical articles, pharmaceutical products, or packaging for food or drink products. Other possible applications are discussed below.

### BACKGROUND TO THE INVENTION

X-ray generators are often used in manufacturing or packaging facilities to sterilise medical articles, pharmaceutical products, or packaging for food or drink products.

In such applications, such as the sterilisation of packaging, conventionally the article to be sterilised is exposed to X-ray radiation produced by means of a radioactive source such as radioactive cobalt. Such radiation comprises “hard” X-rays, i.e. radiation having a high energy, measured in millions of electron volts (eV).

“Hard” X-rays are typically produced by a radioactive decay process, when nuclei undergo a transition into a different element of the periodic table, simultaneously emitting energy through electromagnetic waves. This happens in so-called “Gamma-factories”, which utilize the decay of radioactive cobalt and emit high-energy X-ray photons (in this particular case called gamma-particles, although this is still X-ray radiation, just having a specific energy or wavelength).

Current sterilisation standards require a dosage of the order of 25 kGy (kilograys) to achieve efficient destruction of bacteria to an acceptable level. Such a dosage requires exposure of the packaging to a radioactive source for a prolonged period of time, usually several hours. For this to be practicable, such sterilisation is generally carried out in batches comprised of one or more pallet-loads of products. This is possible because the “hard” X-rays, by virtue of their high energy, have the ability to penetrate deep into a large stack of packages.

However, more recently, as discussed in GB 2444310 A, it has been found that low energy or “soft” X-rays can be better suited to the sterilisation of surfaces. “Soft” X-rays are characterised by being of relatively low energy, with quantum energies predominantly in the range of 5 to 20 keV. Because of their low energy, these soft X-rays have higher absorption. As a result, the efficiency of the X-rays in destroying bacteria on the surface is high and the total exposure that is required may be lower that when using high energy X-rays. The lower X-ray dosage is also desirable in that it reduces the risk of damaging the material being sterilised, and the softness of the X-rays also allows them to be used safely in a production line without risk to personnel or the need for extensive lead shielding.

Such “soft” X-rays can be generated using a particle accelerator (for example, an electron gun) to generate a flow of charged particles. When these particles are decelerated due to the interaction with matter—for example, when they hit a metal target—they emit electromagnetic radiation. If the initial energy of the particle beam is sufficiently high, the electromagnetic radiation is located in the X-ray range of the emission spectrum.

Electrons also emit X-rays when they change the direction of their motion, as in the case of synchrotrons (synchrotron radiation can be generated in a broad spectral range, including X-rays).

In the present work, we deal with the acceleration-based approach to soft X-ray production. The majority of X-ray sources operate at low-power levels (as these are medical/dental X-ray apparatuses, non-destructive testing (NDT) and luggage screening equipment). This is determined by the specific tasks of the equipment: they have to produce an X-ray beam which will assure the best possible image quality. Also, these sources are typically generated as a point-like source, due to the requirements of X-ray imaging. The best way to create such beams for imaging applications is to accelerate electrons in a vacuum and then to direct them onto a metal target.

However, vacuum-based X-ray tubes with heated cathodes are not well suited for long periods of heavy duty operation, as would be the case with sterilisation applications. Therefore, in the present work, we have chosen an approach to X-ray production based on the generation of electrons in gas-filled devices with cold cathodes, rather than in vacuum-based electron beam sources with heated filaments.

Existing “soft” X-ray generator systems, such as the one disclosed in GB 2444310 A, suffer from a number of disadvantages, at least in part resulting from the arrangement of the cathode and anode electrodes and the occurrence of arcing between them. Arcing between the electrodes, and the consequent erosion of the electrodes, leads to a decrease in the operational lifetime of the system. It also affects its ability to produce stable, reliable and reproducible X-rays homogeneously over a large cross-section of the emitter head, and to provide continuous and consistent operation.

Embodiments of the present invention therefore seek to achieve one or more of the following: (1) to increase the operational lifetime of the X-ray generator system; (2) to enable it to operate over a large cross-section of the X-ray emitter head; (3) to improve stability and reproducibility (minimizing pulse to pulse variation of energy generated) of the device; and (4) to avoid arcing and discharge instabilities which reduce the reliability of the device when used in industrial settings requiring high levels of continuous and consistent operation.

### SUMMARY OF THE INVENTION

According to a first aspect of the present invention there is provided an X-ray source as defined in claim 1 of the appended claims. Thus, there is provided an X-ray source for producing soft X-rays, the X-ray source comprising: a cathode having an electron-emitting structure supported by a support structure, the electron-emitting structure being at least partially transparent to X-rays within a region bounded by the support structure; an anode having an X-ray emitting surface parallel to the electron-emitting structure of the cathode; and an electrically insulating spacer arranged between the anode and the cathode; wherein the electron-emitting structure of the cathode and the X-ray emitting surface of the anode are arranged such that, in use, the electron-emitting structure is operable to bombard the anode with electrons, causing X-rays to be emitted from the X-ray emitting surface and to pass through the cathode; and wherein the insulating spacer is arranged between the anode and the support structure of the cathode and projects beyond the support structure, across part of the anode, into the said region.

The expression “region bounded by the support structure” as used above and herein should be interpreted broadly, to encompass an arrangement in which the support structure is present on only two opposing sides of the region in question, as well as arrangements in which the support structure substantially or completely surrounds the region in question.

By virtue of the insulating spacer projecting beyond the cathode support structure, into the said region, across part of the anode, this avoids or at least mitigates the formation of places in the vicinity of the cathode and anode where the electric field strength could otherwise rise substantially. In effect, the insulating spacer “smoothes” the electric field distribution in the vicinity of the anode and cathode. This reduces the probability of electric breakdown between the cathode and anode electrodes, thereby reducing the likelihood of arcing between the electrodes, and reducing the occurrence of erosion of the electrodes. As a consequence, this increases the operational lifetime of the X-ray generator system, makes it more usable for continuous and consistent operation at elevated power levels, enables it to deliver a more homogeneous discharge over a large cross-section of the X-ray emitter head, and improves the overall stability, reliability and reproducibility of the device.

In a presently-preferred embodiment the distance of projection of the insulating spacer beyond the cathode support structure, into the said region, is about 15 mm. This has been found to give optimum results.

Preferably the width of the X-ray emitting surface not covered by the insulating spacer is in the range of about 3 cm to about 10 cm.

Preferably the thickness of the insulating spacer is about 2 mm.

Preferably the insulating spacer is made of a ceramic material such as alumina (Al₂O₃). However, other insulating materials (in particular, other ceramics) may be used instead.

Preferably the electron-emitting structure of the cathode has a grid or mesh structure. Particularly preferably the geometric transparency of the grid or mesh structure is about 70% to 80%.

Preferably the X-ray source further comprises an X-ray transparent window on the opposite side of the cathode from the anode, the window defining a chamber between the window and the anode. In the presently-preferred embodiment this chamber contains a gas at sub-atmospheric pressure. The gas may be an inert gas such as helium or nitrogen, or may be air. Preferably a molecular sieve is provided between the gas supply and the chamber, to prevent moisture or dust etc. from entering the chamber. A vacuum pump may also be provided in communication with the chamber, to achieve and maintain sub-atmospheric pressure within the chamber.

Preferably the X-ray transparent window comprises Kapton™ (RTM), as this has been found to have advantageous properties (including becoming stronger when exposed to X-rays, whereas other materials can break down or become brittle over time).

Particularly preferably the window is formed of an electrically conductive material, or further comprises a coating formed of an electrically conductive material. This enables the window to be held at the same electric potential as the electron-emitting structure of the cathode, thereby preventing charged particles from the cathode being accelerated towards the window and damaging it. Accordingly, the window may advantageously be electrically connected to the electron-emitting structure of the cathode.

Preferably the anode is formed of a metal block, at least several millimetres in thickness.

Preferably the anode further comprises cooling means, such as one or more cooling pipes in thermal communication with the anode.

Preferably the electron-emitting structure of the cathode is at least partly formed of copper. Preferably the anode is also at least partly formed of copper. For example, it may be formed of bulk copper, or iron coated with copper. Although copper is our presently-preferred material for the cathode and anode, other materials may be used instead, provided they have characteristic emission lines in the spectral range below 10-12 keV.

The X-ray source may further comprise a power supply cable electrically connected to the anode.

Preferably the X-ray source further comprises isolation material configured to match the wave impedance of the power supply cable with the wave impedance of the anode. This advantageously reduces reflections of the voltage pulses applied to the emitter head.

Preferably the electron-emitting structure of the cathode is at ground potential.

Preferably the electron-emitting structure of the cathode is electrically connected to the cathode support structure, enabling the electron-emitting structure and the cathode support structure to be held at a common potential.

Preferably the cathode support structure is connected to, or integrally formed with, a housing structure for the X-ray source. The housing structure may be arranged around at least part of the anode.

Preferably the insulating spacer extends between the housing structure and the anode.

Preferably the X-ray source further comprises means for generating a voltage between the anode and the cathode.

Particularly preferably the means for generating a voltage comprises inductive energy storage means. With such an arrangement, rising current results in a voltage rise on the inductors, thus effectively reducing the voltage applied to any spark that may be created within the X-ray generator—effectively functioning as a self-damping limiter. This further improves the operational stability and longevity of the X-ray device.

Preferably the means for generating a voltage is configured to supply high-voltage short-duration pulses to the anode.

Preferably the X-ray source is configured to emit X-rays with quantum energies in the range of 5 keV to 20 keV, although this energy may be increased if required by a particular application.

According to a second aspect of the invention there is provided sterilisation apparatus comprising an X-ray source in accordance with the first aspect of the invention.

According to a third aspect of the invention there is provided a production line or a manufacturing or packaging facility comprising sterilisation apparatus in accordance with the second aspect of the invention.

According to a fourth aspect of the invention there is provided a method of sterilising an article, the method comprising irradiating the article with X-ray radiation using an X-ray source in accordance with the first aspect of the invention. The article being irradiated may be, for example, a medical article, a pharmaceutical product, packaging material for a food or drink product, a plastic film, a blood sample, or a foodstuff or beverage.

According to a fifth aspect of the invention there is provided an outcoupling window for an X-ray source, the window comprising a material that is at least partially transparent to X-rays; wherein the window is formed of an electrically conductive material or further comprises a coating formed of an electrically conductive material.

### BRIEF DESCRIPTION OF THE DRAWINGS

Embodiments of the invention will now be described, by way of example only, and with reference to the drawings in which:

FIG. 1 is a schematic cross-sectional diagram of an X-ray generator according to an embodiment of the present invention;

FIG. 2 illustrates X-ray transmission data for a Kapton™ window of the X-ray generator (the plotted data being X-ray transmission data in respect of Kapton™ polyimide film 75 μm thick);

FIG. 3 is a plot of mass-energy absorption coefficient versus photon energy for typical plastic packaging material (of density 1 g cm⁻³);

FIG. 4 is a plot of dose efficiency as a function of photon energy;

FIG. 5 illustrates the generation of X-rays by an electron beam impinging on a metal target;

FIGS. 6a, 6b and 6c provide a comparison of theoretical and experimental bremsstrahlung spectra;

FIG. 7 is a plot showing the intensity of Cu Kα characteristic radiation;

FIG. 8 is a schematic diagram of production line apparatus in which a plastic film is sterilised with X-rays;

FIG. 9 is a plot showing specific dose-rate distribution along a plastic film; and

FIG. 10 illustrates representative data for the dose-area integral for a copper anode X-ray source, calculated for different distances and voltages.

### DETAILED DESCRIPTION OF PREFERRED EMBODIMENTS

The present embodiments represent the best ways known to the applicants of putting the invention into practice. However, they are not the only ways in which this can be achieved.

### Overview of Presently-Preferred Embodiment

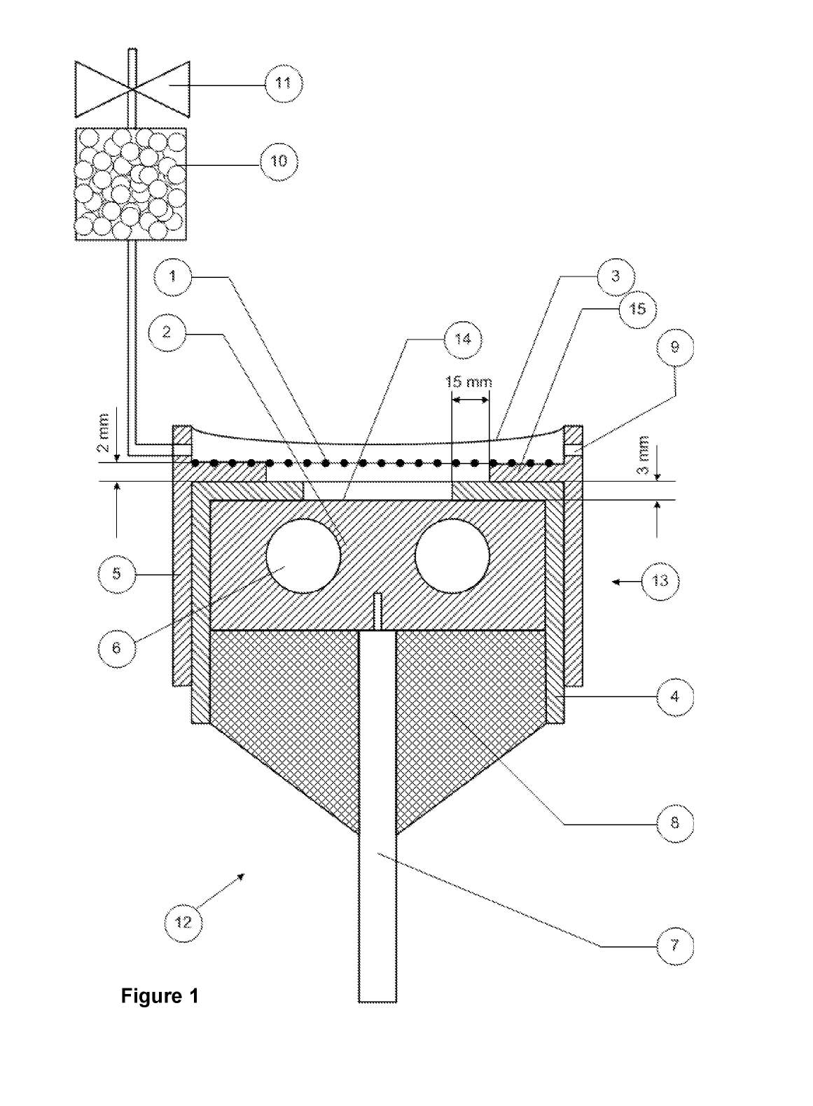

FIG. 1 illustrates an X-ray generator 12 comprising a gas-filled flash X-ray tube with inductive energy storage for the sterilisation of products such as plastic medical articles. The emitter head 13 of the X-ray tube comprises a cold cathode 1 made of a highly transparent metal grid or mesh, and an anode 2 made of massive metal energized by high-voltage short-duration pulses. Electrons emitted by the grid cathode 1 strike the metal anode 2 and generate characteristic and bremsstrahlung X-ray radiation from the emission surface 14 of the anode 2. The X-ray radiation passes through the cathode grid 1 and irradiates the article(s) to be sterilised.

An electrically insulating (preferably ceramic) spacer 4 provides means to avoid shorting or arcing of the anode-cathode discharge gap upon application of pulsed power, thereby achieving increased operational lifetime and more stable and reproducible operation, while also creating conditions for generating an X-ray beam over a large area.

Furthermore, in the presently-preferred embodiment, a power supply based not on capacitive energy storage but on inductive energy storage is used.

It should be noted that the diagram in FIG. 1 is not to scale. Furthermore, the measurements included in this diagram relate only to a presently-preferred embodiment, and are by way of example only; in alternative embodiments the constituent features may have different measurements. The component regions on the left side of FIG. 1 predominantly mirror those on the right; for clarity each component has been labelled only once.

### Detailed Description of X-ray Generator

FIG. 1 illustrates an X-ray generator 12 according to a presently-preferred embodiment of the present invention. A homogeneous X-ray beam is generated from an irradiator with a large cross-section area, rather than from a point source. A wide range of shapes and dimensions of the emitter head 13 are possible. For example, it may be long and thin (e.g. extending with uniform cross-section normal to the plane of FIG. 1), round or square, or any other shape—depending upon the requirements dictated by the shape of the target being irradiated.

As illustrated, the electrode system of the X-ray generator 12 comprises a cathode 1 and an anode 2. The cathode 1 has a grid or mesh electron-emitting structure (as described in more detail below; a number of different metals which possess good heat and electric conductivity may be used). The cathode 1 is shaped and configured such that X-rays can penetrate the structure relatively freely. In the presently-preferred embodiment the mesh of the cathode 1 has a geometric transparency of about 70% to 80% (below this range it will work less efficiently, lowering the energy yield conversion into X-rays, while above this range the mesh may become too fragile and break).

The anode 2 is made of a metal block at least several millimetres in thickness, which provides the possibility for enhanced cooling of the anode 2 and heat removal via cooling pipes 6. This is important for stable and continuous operation of the device in a real operating environment. The metal block may be cooled using a wide range of cooling systems employing a heat exchange. For example, a water based cooling system operating at a rate of 1 litre/second would be sufficient to dissipate 200 kW of heat energy absorbed by the metal block.

The preferred material for the electrodes 1, 2 is copper, due to the fact that copper emits a strong line of characteristic radiation Cu K—the first characteristical K emission line of copper in a low-energy (8 keV) part of the X-ray spectrum. However, it is possible to make the electrodes 1, 2 from other metals or conductive materials, of which their surface can be covered with a thin copper layer to provide similar emission properties to bulk copper. It is preferable that both cathode 1 and anode 2 consist of, or have their surface covered with, similar material in order to avoid an eventual change in the emission spectrum properties due to changes of the surface composition in the case of spattering, which can occur if the electrodes 1, 2 are composed of different materials. Copper is our presently-preferred material for the electrodes 1, 2; however, other materials may be used instead, provided they have characteristic emission lines in the spectral range below 10-12 keV.

The gap between the cathode 1 and the anode 2 is filled with gas at sub-atmospheric pressure (low or intermediate pressure). It can be a specially selected inert gas, such as helium or nitrogen, but alternatively normal air can be used to fill in the device. Gas pressure inside the device can be controlled with an external vacuum pump connected to the device through an opening 9. To fill in the gas, an opening from the opposite side of the vessel is used, which provides a controlled gas leakage through a valve 11. To ensure that no moisture, dust, etc. enters the irradiator, a molecular sieve 10 is placed after the valve.

A working prototype has been successfully demonstrated using a discharge in air at a pressure of 5 mbar. However, as mentioned earlier, other gases may be used, which would allow embodiments to operate at different pressures.

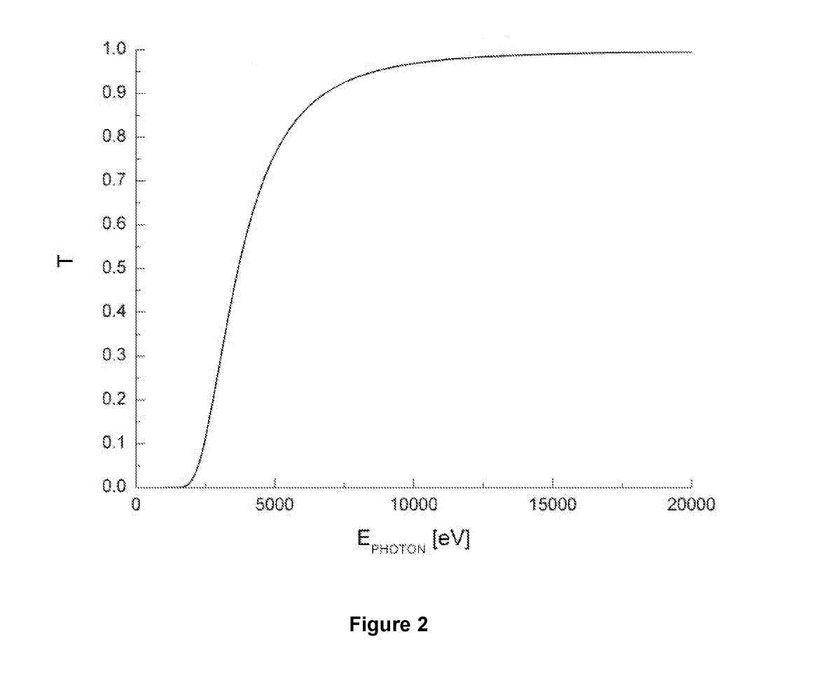

Another important part of the system is an outcoupling window 3, which forms a chamber between the anode 2 and the window 3 in which the above-mentioned gas is contained, and also encloses the cathode 1. In the presently-preferred embodiment this window 3 is made of a polyimide film, preferably Kapton™. Although other materials may be used, to date Kapton™ is the best we have found, as it demonstrates some particularly attractive features in this application, as it becomes stronger when exposed to X-rays, while other materials can break down or become brittle over time. Ideally the window 3 should meet several requirements: it should withstand the pressure difference and not break, have low absorption losses for X-rays (see transmission data in FIG. 2), and should not lose its strength and transparency under the influence of intense X-ray irradiation. Materials other than Kapton™ can be used for the outcoupling window 3, provided they have material properties and transmission characteristics similar to those of Kapton™, or better, although at present we are not aware of any such material.

The window 3 is preferably formed of an electrically conductive material, or the inner surface of the window 3 may be covered with a thin layer of electrically conductive material. For example, a layer of conductive material such as graphite may be deposited on the inner surface of the window 3. In our presently-preferred embodiment, however, a commercially available electrically-conductive polyimide film, Kapton™ RS, is used to form the window 3. Kapton™ RS comprises a polyimide film loaded with conductive carbon. By making the window 3 electrically conductive, this enables the window 3 to be kept at the same electric potential as the grid or mesh of the cathode 1, thereby preventing an electric field from “hanging” between the cells of the cathode grid/mesh in the direction of the outcoupling window 3 (which would result in a constant flow of accelerated electrons towards the window 3, resulting in sputtering of the window material and causing it to be damaged).

The cathode 1 is mounted on, and electrically connected to, a metal support structure 15 which is kept at ground potential. Thus the cathode 1 has similar potential. The cathode support structure 15 is connected to, or integrally formed with, a housing structure 5 in which at least part of the anode 2 is mounted. The cathode support structure 15 (and the rest of the housing structure 5) is electrically isolated from the anode 2 by the insulating spacer 4. The outcoupling window 3 is also mounted on the housing structure 5, over the cathode 1. If, as in the presently-preferred embodiment, the outcoupling window 3 is electrically conductive, then the outcoupling window 3 is electrically connected to the housing structure 5 and the cathode support structure 15, so that the window 3 is at the same electric potential as the cathode 1.

The cathode support structure 15 and/or the housing structure 5 may be formed of stainless steel, or any other suitable material.

High-voltage pulses are supplied by a power supply to the irradiator via a high-voltage cable 7. The power supply is preferably a high-voltage generator with inductive energy storage. The latter is important for stable device operation, and the reasons for this are explained below.

A bulk piece of isolation material 8 matching the wave impedance of the power cable 7 with the wave impedance of the emitter head 13 serves effectively as a transformer, that reduces the reflections of the voltage pulses applied to the emitter head 13.

Insulating (e.g. Ceramic) Spacer

Although, in the presently-preferred embodiment described below, the insulating spacer 4 is made of a ceramic material (e.g. alumina), in alternative embodiments it can be made of other insulating materials instead.

The ceramic spacer 4 serves to insulate the anode's emitter surface 14 from the cathode support structure 15 and the metal housing 5, and simultaneously improves the operational stability of the emitter. In order to achieve this improvement in stability, we have decreased the size of the emitter surface 14 by making the opening provided by the ceramic spacer 4 slightly smaller than the opening provided by the cathode support part of the housing 15. In the example shown in FIG. 1, this difference is 15 mm of additional ceramic material that projects across the surface of the anode 2. Regarding this distance by which the ceramic spacer 4 projects across the anode 2, distances shorter than 15 mm were tested, with unsatisfactory results. Distances larger than 15 mm will result in effective operation, but would reduce the area of X-ray emission, and hence the yield. Thus, in the presently-preferred embodiment, the distance by which the ceramic spacer 4 projects across the anode 2 is about 15 mm.

By having this ceramic spacer 4 we avoid the formation of places in the vicinity of the cathode and anode electrodes 1, 2 where the electric field strength could rise substantially. In effect, the ceramic spacer 4 “smoothes” the electric field distribution in the vicinity of the electrodes 1, 2. In X-ray sources known in the art, which have places in which the electric field strength can rise substantially, there is a substantial chance that there will be a short electric breakdown between the electrodes, resulting in arcing of the charge and a disruption of the X-ray generation. The result would be the erosion of the electrodes and subsequent deterioration of the inner side of the device.

As illustrated, the insulating ceramic spacer 4 preferably also extends downwards, between the housing structure 5 and the sides of the anode 2, as well as projecting across the surface of the anode 2. Thus the housing structure 5 is electrically isolated from the anode 2.

The thickness of the ceramic spacer 4 is preferably about 3 mm, as illustrated. The ceramic spacer 4 is preferably fitted in contact with the upper surface of the anode 2, and in contact with the underside of the cathode support structure 15 and the inner surface of at least part of the housing 5.

The width of the X-ray emitting surface 14 of the anode 2 exposed between opposing edges of the ceramic spacer 4 is preferably in the range of about 3 cm to about 10 cm. The X-ray beam produced from the exposed X-ray emitting surface 14 is homogeneous and well-directed.

As mentioned above, although the spacer 4 of the presently-preferred embodiment is made of a ceramic material (e.g. alumina), in alternative embodiments other insulating materials can be used instead.

Our experience shows that in the case of a regular design of the electrode, when there is no protective insulating spacer 4 (not necessarily ceramics), once in every 10,000 pulses a spark due to discharge instabilities might occur. At power levels higher than several kW, more thermally stable materials would be used; however, not a material such as Teflon™, as it is too isolating of the charge. Given that our device can operate at a repetition rate of up to 20 kHz, this effectively would mean that without these protective means the device might be suited for short-term scientific research but would be completely unfit for routine industrial operation. However, the protective insulating spacer 4 enables the present X-ray generator to be employed in long-term continuous operation, such as on a production line in a manufacturing or packaging facility.

Another important feature of our system is a combination of two protective means. One is the above-mentioned specially shaped ceramic spacer 4, and the other is the use of a power supply based not on capacitive energy storage but on inductive energy storage. The difference here occurs due to the following effects:

In a capacitive storage device if an accidental breakdown occurs, it is in no way affected by the power supply itself, and can develop a full-blown electric spark that would damage the surface of the electrodes and the device itself. However, by employing an inductive energy storage power supply, rising current results in a voltage rise on the inductors, thus effectively reducing the voltage applied to a spark. In effect, it functions as a self-damping limiter. Together with the ceramic protector 4, this substantially improves the operational stability and longevity of the device.

There is also an ancillary but beneficial by-product from the present system, namely UV radiation generation within the chamber between the cathode 1 and anode 2, which ensures a stable and sterile environment within the X-ray device.

### Summary of Components in the Apparatus of FIG. 1, with Example Specifications

1. Cathode grid (preferably copper) having a geometric transparency of about 70-80%
2. Anode (preferably copper, or iron covered with copper)
3. Output window (e.g. Kapton™ 200RS100)
4. Insulating spacer (e.g. alumina ceramic)
5. Housing (e.g. stainless steel) at ground potential
6. Cooling pipes (suitable cooling liquids are, for example, transformer oil or silicone oil)
7. High voltage cable
8. Isolation matching the wave impedance of the cable with the emitter head
9. Port for pumping gases (e.g. dry air or nitrogen, at 3-10 mbar)
10. Cartridge with molecular sieve (e.g. X13)
11. Regulated leakage valve
12. X-ray generator
13. Emitter head
14. Emitter surface
15. Cathode support structure (e.g. stainless steel)

### Soft X-rays in the Grenz-Ray Region

The following section provides further details on the soft X-rays generated by the embodiments described above, and their use in sterilisation applications.

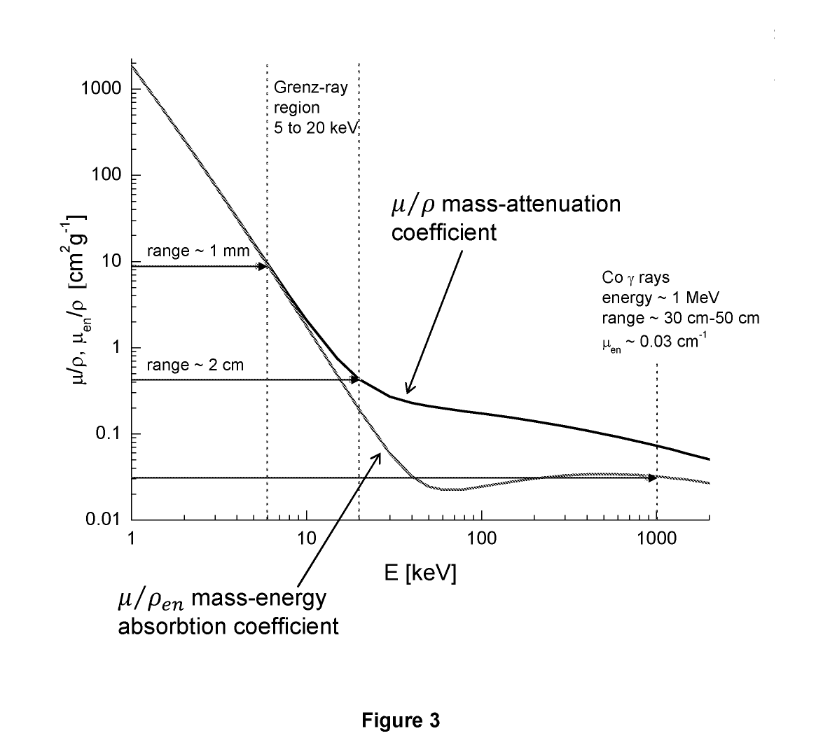

The main idea in our approach to the sterilisation of organic matter lies in the use of soft X-rays with quantum energies predominantly in the interval 5 to 20 keV instead of high energy gamma sterilisation using Co⁶⁰. The possible advantages and drawbacks of this approach can be seen from FIG. 3 where the mass-energy absorption coefficient is presented for a typical plastic used in packaging (of density 1 g cm⁻³), together with the mass-attenuation coefficient. In the graph, the upper line is the mass-attenuation coefficient, and the lower line is the mass-energy absorption coefficient.

In the graph we have explicitly marked two different energy regions. One region corresponds to the high energy gamma rays (“hard” X-rays) produced by Co⁶⁰ and lies near 1 MeV, while the other region (also called the Grenz-ray region) is limited within 5 to 20 keV and corresponds to “soft” X-rays. The lower limit of the Grenz-ray region is due to a small photon range (<1 mm) for energies less than ˜5 keV. The photons with smaller energy cannot escape a traditional X-ray tube due to strong absorption in the vacuum window. The upper limit of the Grenz-ray region is determined by the change of mechanism by which X-rays interact with matter. At energy less than ˜20 keV photons interact with matter predominantly via photoelectric absorption while the scattering plays minor role. At higher energies the mechanism changes to Compton scattering while photoelectric absorption is of no importance.

The main parameter which determines the effectiveness of sterilisation is the dose. The dose is the energy of X-rays absorbed by a unit mass of matter. It is instructive to compare the doses produced by X-rays with different energies. The dose-rate, produced by the flux F [ph cm⁻²s⁻¹] of photons having energy E, is equal to the product

$$FE ( \frac{{μ}_{en}}{ρ} ) .$$

Suppose we generate equal fluxes of photons with different energies. Then the photons with different energies E_{low}, and E_{high} will have the same “dose efficiency” if the products

$${δ}_{low} = {( \frac{{μ}_{en}}{ρ} )}_{low} {E}_{low} and {δ}_{high} = {( \frac{{μ}_{en}}{ρ} )}_{high} {E}_{high}$$

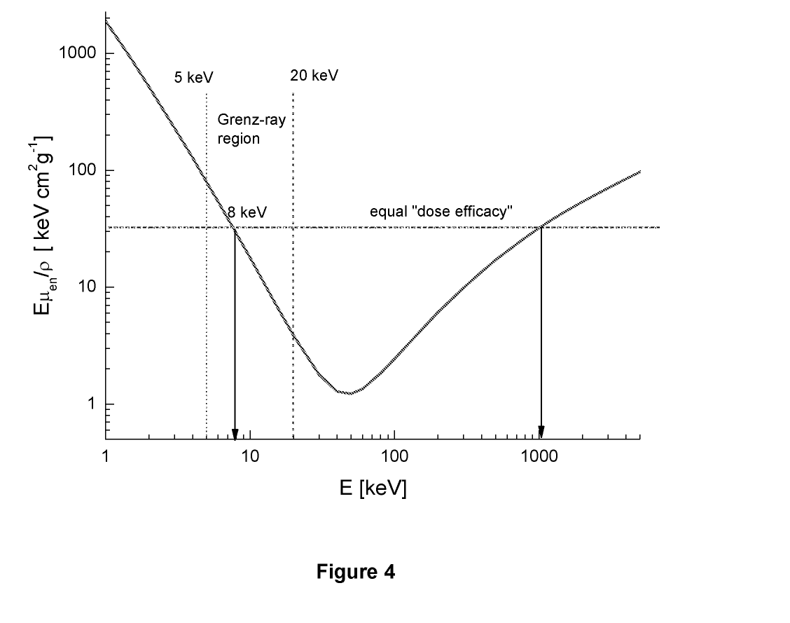

are equal. The data for δ versus photon energy are plotted in FIG. 4.

Surprisingly, there is mirror-like correspondence in dose efficiency between the low and high energy (“soft” and “hard” X-ray) regions. For example, a photon with quantum energy of 8 keV has exactly the same dose efficiency as a photon with energy of 1 MeV. It is interesting to note that the photons with energy close to 50 keV are useless for the purpose of sterilisation. This is due to the deep well on the dose efficiency curve in this energy region, as shown in FIG. 4.

Thus, we came to the conclusion that X-rays in the Grenz-ray region near 10 keV have the same dose efficiency as more energetic 1 MeV photons. The advantage of sterilisation with low energy X-rays becomes clearer if we compare the energy required to produce the same dose with low and high energy X-rays.

As the dose efficiencies are equal for energies of 8 and 1000 keV, the photon fluxes should be equal too to produce the same dose-rate. This means that the required power P=E_{low}F of low energy X-rays comprises just 8/1000 of the power of high energy gamma radiation. Suppose we generate low energy X-rays with efficiency of 0.8% (ratio of the output power of X-rays to input electric power). Then the same sterilisation effect will be achieved as with 1 MeV gamma rays if they were generated with 100% efficiency.

Sterilisation with low energy X-rays has a potential drawback, however. The range of low energy photons is relatively small—around 1-20 mm in plastics and water in the Grenz-ray region. It should be noted that, with plastics, there may be several layers, and the overall range of penetration of the X-rays in the Grenz-ray region may be more than 20 mm if the structure being irradiated is not solid plastic but contains air (e.g. as in foams, tubing or syringes). Of course, the photon range in atmospheric air is larger than 1 metre, even at the lower boundary of the Grenz-ray region, due to the very small density of air. It follows that there is a natural niche for low energy X-ray sterilisation—thin low density materials, such as medical devices, plastic packaging or blood samples, lettuce and hamburgers.

X-ray Generation by Kilo-Electron Volts (keV) Electrons

In this section we describe a model for X-ray generation by electron beams impinging on metal targets and check the theoretical results against available experimental data. The most important practical result is the calculation of dose-rate at various distances from the target, which can be of use when configuring an implementation of an embodiment of an X-ray generator as described above. This also shows the direct advantage of our irradiation scheme for radiation sterilisation, in comparison to other sterilisation processes.

By definition, the number of X-ray photons, emitted during unit time interval within unit solid angle and unit energy interval, is as follows:

$${S}_{ph} ( \vec{τ} , E , \vec{x} ) = \int d {Ω}_{e} d {E}_{e} J ( \vec{x} , {E}_{e} , {\vec{τ}}_{e} ) \frac{{d}^{2} σ ( {\vec{τ}}_{e} , {E}_{e} , \vec{τ} , E )}{d Ω d E} {N}_{oi} \qquad ( 1 )$$

Here J(\vec{x},Eₑ,\vec{σ}ₑ) is the spectral density of electrons found from the solution of transport equation, introduced above, and

$$\frac{{d}^{2} σ ( {\vec{τ}}_{e} , {E}_{e} , \vec{τ} , E )}{d Ω d E}$$

is atomic field bremsstrahlung cross-section differential in photon energy and angle of emission [1].

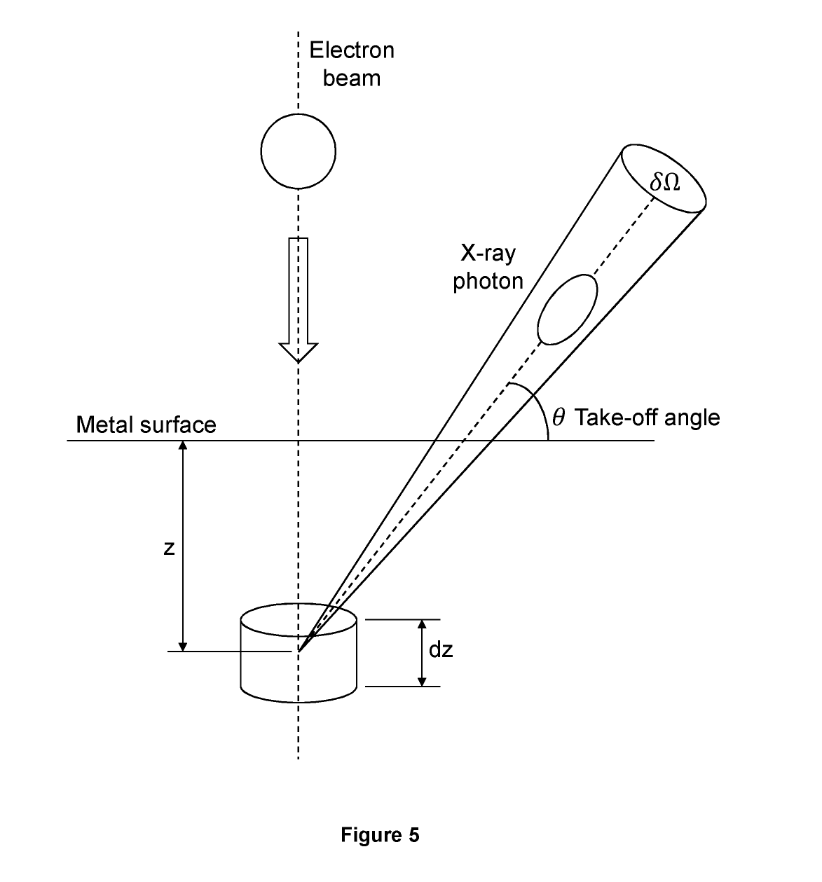

Consider the electron beam impinging normally on a metal target as shown in FIG. 5. The number of photons emitted from the unit area of the target in the direction of take-off angle θ within unit energy and solid angle intervals—spectral brightness—is given by the following relation:

$$B ( \vec{τ} , E ) = {\int }_{0}^{∞} d {zS}_{ph} ( z , E , \vec{τ} ) exp - \frac{μ ( E ) z}{Sin ( θ )} \qquad ( 2 )$$

where μ(E) is X-ray attenuation coefficient of the target metal.

Finally, the dose-rate distribution

$$\frac{d D}{d t} ,$$

produced by an extended X-ray source at the point of observation \vec{x}, is as follows:

$$\frac{d D}{d t} = \int d {Ω}_{source} \int d E [ \frac{{μ}_{air} ( E )}{{ρ}_{air}} ] EB ( \vec{τ} , E ) exp - {μ}_{air} ( E ) L \qquad ( 3 )$$

Here the integral is taken over the solid angle at which the source is seen from the point of observation and L is the distance between the point of observation and the surface area at the source.

Comparison of Theoretical Model with Experimental Data

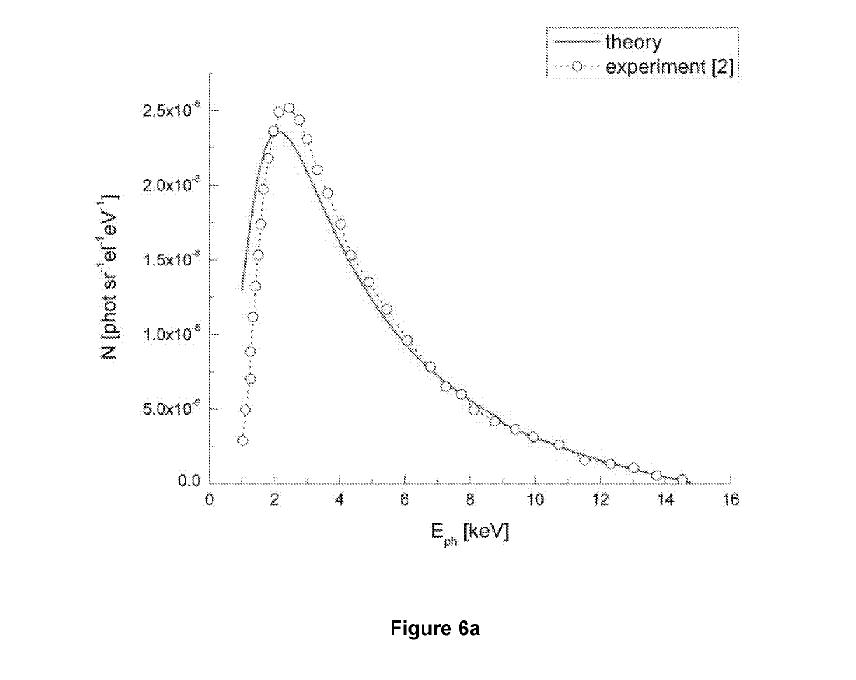

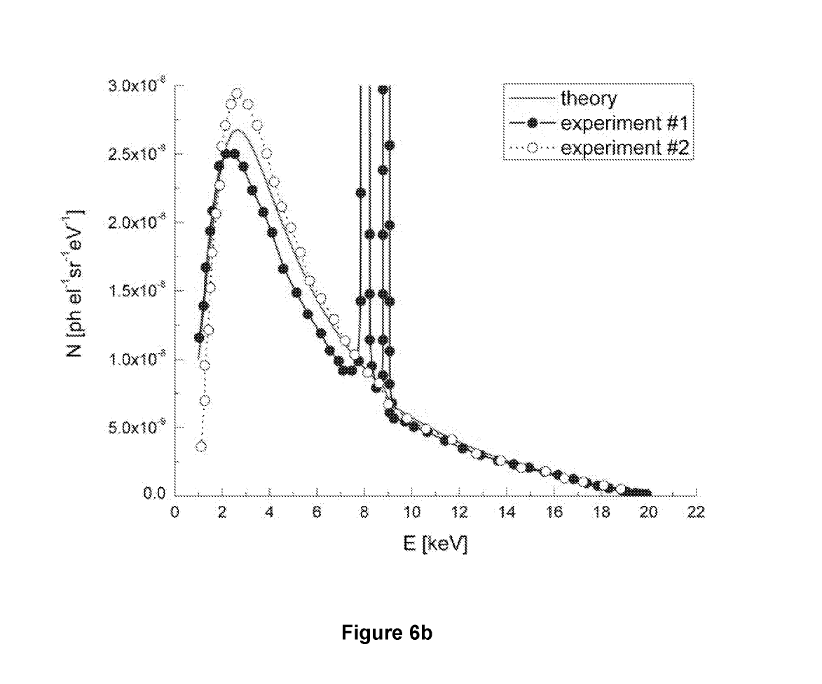

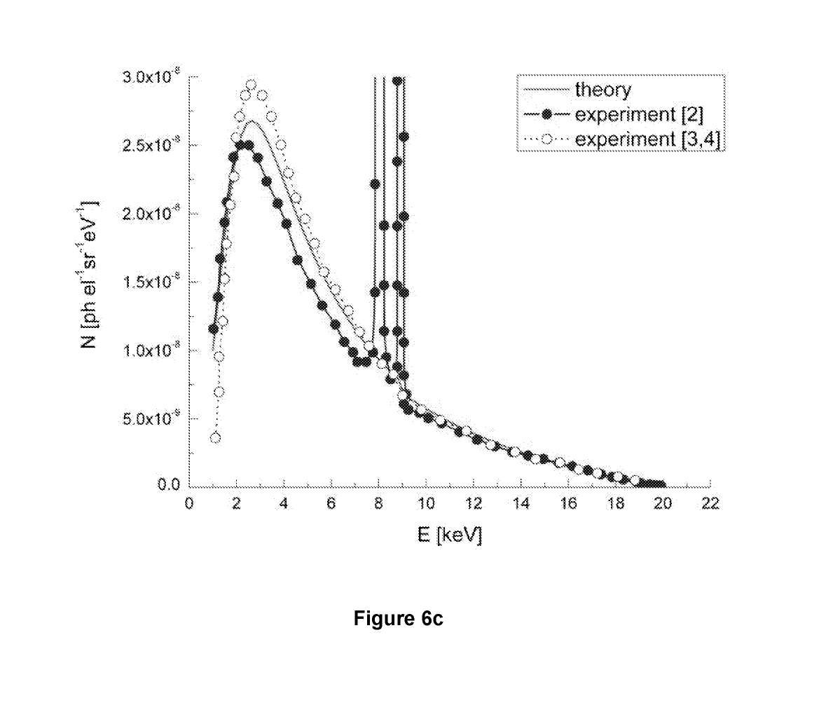

X-ray spectral brightness B(\vec{σ},E) was calculated with the use of relation (2). The results of calculation are presented in FIGS. 6a-6c in comparison with available experimental data [2-4]. The parameters used in FIGS. 6a-6c are as follows:

FIG. 6a: E_{beam}=15 keV, normal incidence, take-off angle=40°.
FIG. 6b: E_{beam}=20 keV, normal incidence, take-off angle=40°.
FIG. 6c: E_{beam}=20 keV, normal incidence, take-off angle=40°.

It is seen that the discrepancy between calculated and measured data is smaller than the experimental error. The theoretical curves lie right in-between the data of different authors.

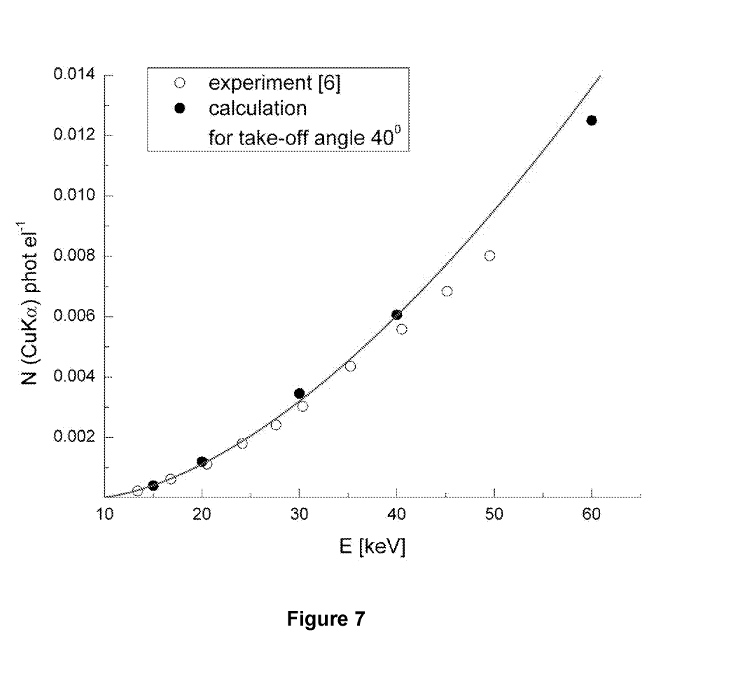

The intensity of characteristic K-radiation was calculated with the use of experimental cross-section[5]. The result of calculation is presented in FIG. 7.

There is also good agreement with experiment [6]. Thus, the model developed in the course of the present work gives reliable spectra of X-rays generated by stopping of kilo-electron volts electron beams in metal targets and can be used for engineering of X-ray sterilisation sources.

### Practical Example—Sterilization of a Plastic Item Using an Extended X-ray Source

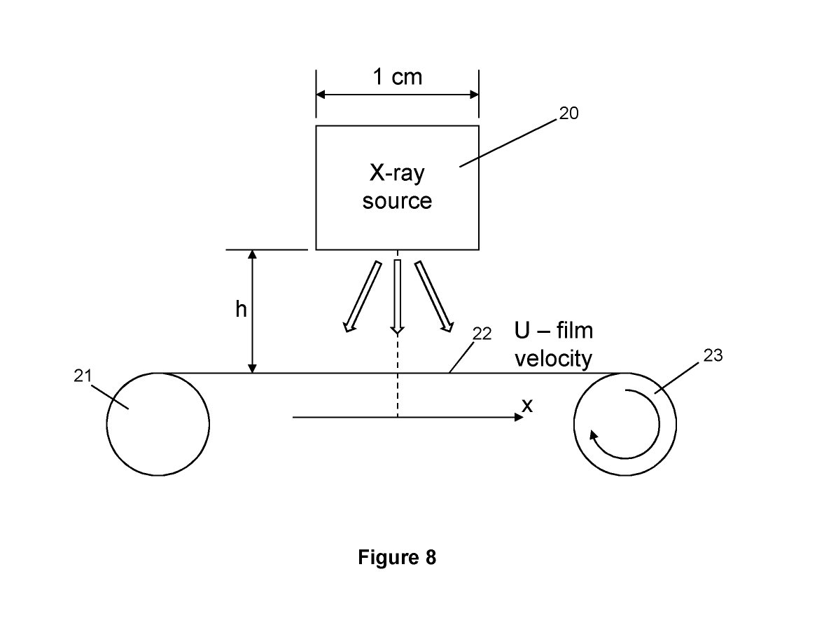

A schematic example of an X-ray sterilization system is shown in FIG. 8. An item to be sterilized (in this case, a plastic film 22) is moved (by rollers 21 and 23) with a velocity U under an X-ray irradiator 20 comprising a rectangular X-ray source with a copper anode. For the purposes of this example, we take the width of the X-ray source to be 1 cm and its length to be 50 cm. The distance between the irradiation unit 20 and the plastic film 22 is denoted by h. The co-ordinate axis x is directed along the film.

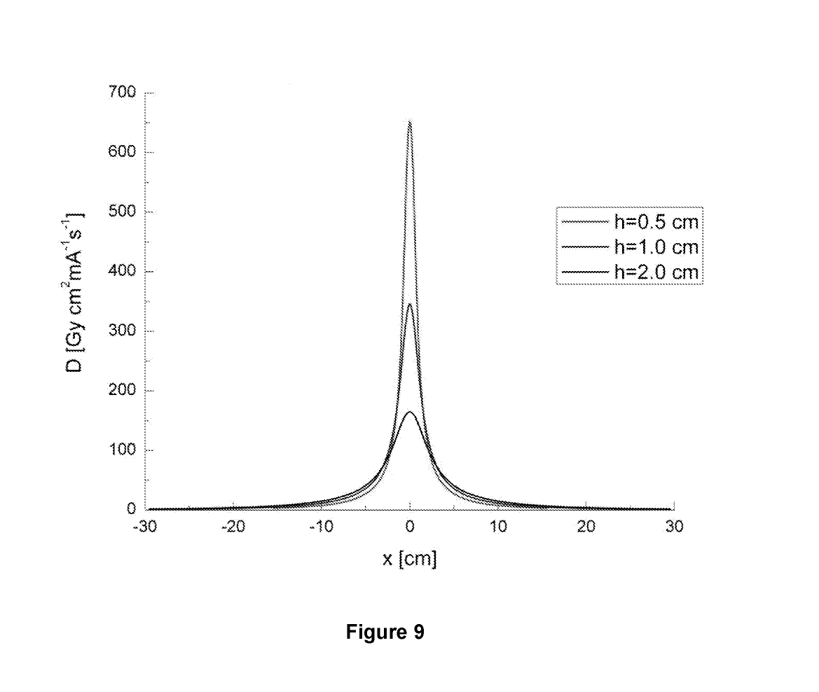

The dose-rate can be calculated with the use of equation (3). The specific dose-rate distribution at different distances h is presented in FIG. 9 for the particular case of the source operating at 60 kV. The plots are based on a dose rate distribution for a rectangular source, with L=50 cm, w=1 cm, U=60 kV, and a copper anode. ‘h’ represents the distance to the anode. In the graph, the uppermost line refers to h=0.5 cm, the intermediate line to h=1.0 cm, and the lowermost line to h=2.0 cm.

The plots in FIG. 9 present the dose received by the item to be sterilized during one second with an X-ray source driven by the electron beam with a current density of 1 mA cm⁻². It is important to note that peak dose-rates achieve very high values of ˜1 kGy s⁻¹ with very modest parameters of X-ray source.

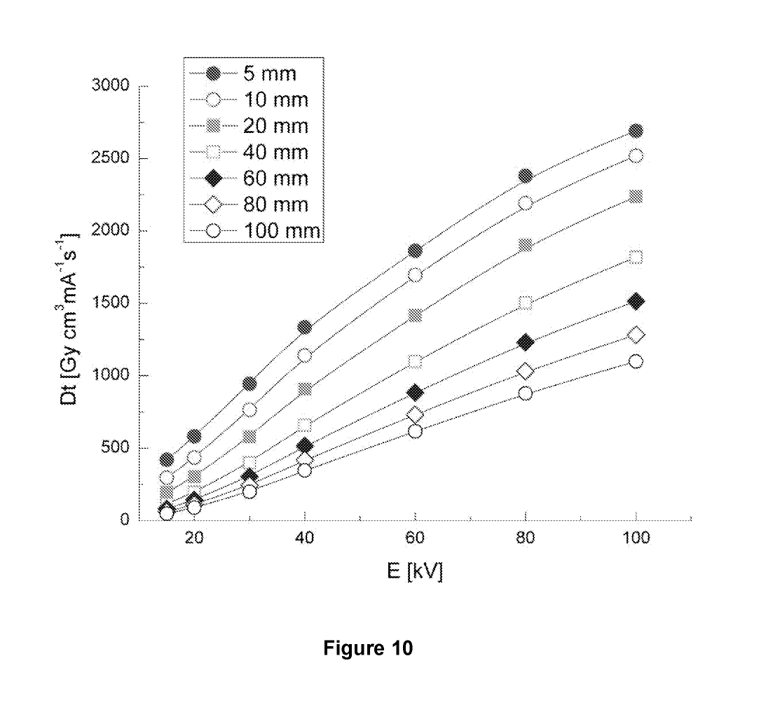

To illustrate our approach, we present the calculations of the dosage delivered to a plane plastic sample during the passage of the irradiation area. The calculation of the dosage received by the item during the passage of the irradiation area is done with the use of the following relation:

$$Dosage = \frac{\int d xD}{U} J \qquad ( 4 )$$

where J is the electron beam current density. U is the conveyor belt velocity, and dose-area integral ∫d×D is taken over the length of the object. The representative data for dose-area integral, calculated for different distances and voltages, are presented in FIG. 10.

The electric energy W required for sterilisation of unit surface area of the item which is sterilized is given by the following relation:

$$W = \frac{{E}_{beam} {Dosage}_{th}}{\int d xD} \qquad ( 5 )$$

Here E_{beam} is electron beam energy in keV, Dosageₜₕ=2500 Gy is the minimal dosage required for log 6 reduction of bioburden, and dose-area product is in Gy cm² mA⁻¹s⁻¹. Suppose the X-ray source operates at 60 kV and the source-object separation is 2 cm. Then, as follows from the data in FIG. 10, the required energy is:

$$W = \frac{60 \times 2500}{1300} = 115.4 J {cm}^{- 2} \qquad ( 6 )$$

### REFERENCES

[1] L. Kissel, C. A. Quarles, R. H. Pratt. Shape functions for atomic-field bremsstrahlung from electrons of kinetic energy 1-500 keV on selected neutral atoms 1<Z<92. Atomic data and nuclear data tables 28, 381-460n (1983)
[2] Z. J. Ding, R. Shimizu, K. Obori, Monte Carlo simulation of X-ray spectra in electron probe microanalysis: Comparison of continuum with experiment. J. Appl. Phys. 76, 7180-7187 (1994)
[3] F. Salvat, J. M. Fernandez-Varea, J. Sempau et. al, Monte Carlo simulation of bremsstrahlung emission by electrons. Rad. Phys. Chem. 75, 1201-1219 (2006)
[4] E. Acosta, X, Llovet, E. Coleoni et. al. Monte Carlo simulation of X-ray emission by kilovolt electron bombardment. J. Appl. Phys. 83, 6038-6049 (1998)
[5] X. Llovet, C. Merlet, F. Salvat. Measurements of K-shell ionization cross-sections of Cr, Ni and Cu by impact of 6.5-40 keV electrons, J. Phys. B: At. Mol. Opt. Phys. 3761-3772 (2000)
[6] V. Metchnik, S. G. Tomlin. On the absolute intensity of characteristic radiation. Proc. Phys. Soc. 81, 956-964 (1963)

1. An X-ray source for producing soft X-rays, the X-ray source comprising:
a cathode having an electron-emitting structure supported by a support structure, the electron-emitting structure being at least partially transparent to X-rays within a region bounded by the support structure;
an anode having an X-ray emitting surface parallel to the electron-emitting structure of the cathode; and
an electrically insulating spacer arranged between the anode and the cathode;
wherein the electron-emitting structure of the cathode and the X-ray emitting surface of the anode are arranged such that, in use, the electron-emitting structure is operable to bombard the anode with electrons, causing X-rays to be emitted from the X-ray emitting surface and to pass through the cathode; and
wherein the insulating spacer is arranged between the anode and the support structure of the cathode and projects beyond the support structure, across part of the anode, into the said region.

2-51. (canceled)

52. A method of sterilizing an article, the method comprising:
configuring a cathode of an X-ray source with an electron-emitting structure;
supporting the electron-emitting structure by a support structure;
configuring the electron-emitting structure to be at least partially transparent to X-rays within a region bounded by the support structure;
configuring an anode of the X-ray source with an X-ray emitting surface parallel to the electron-emitting structure of the cathode;
arranging an electrically insulating spacer between the anode and the cathode by arranging the electrically insulating spacer between the anode and the support structure of the cathode, and by projecting the electrically insulating spacer beyond the support structure, across part of the anode, and into the said region;
emitting soft X-rays from the X-ray emitting surface by bombarding the anode with electrons from the electron-emitting structure, and passing the soft X-rays through the cathode; and
irradiating the article with the emitted soft X-rays to sterilize the article.

53. An article sterilized by performing the following steps:
configuring a cathode of an X-ray source with an electron-emitting structure;
supporting the electron-emitting structure by a support structure;
configuring the electron-emitting structure to be at least partially transparent to X-rays within a region bounded by the support structure;
configuring an anode of the X-ray source with an X-ray emitting surface parallel to the electron-emitting structure of the cathode;
arranging an electrically insulating spacer between the anode and the cathode by arranging the electrically insulating spacer between the anode and the support structure of the cathode, and by projecting the electrically insulating spacer beyond the support structure, across part of the anode, and into the said region;
emitting soft X-rays from the X-ray emitting surface by bombarding the anode with electrons from the electron-emitting structure, and passing the soft X-rays through the cathode; and
irradiating the article with the emitted soft X-rays to sterilize the article.
